(19) United States
(12) Patent Application Publication
(10) Pub. No.: US 2025/0037861 A1
(43) Pub. Date: Jan. 30, 2025

(54) FEDERATED LEARNING OF MEDICAL VALIDATION MODEL

(71) Applicant: Roche Diagnostics Operations, Inc., Indianapolis, IN (US)

(72) Inventors: Yi YAO, Shanghai (CN); Wei Bin XING, Shanghai (CN); Xiao Jun TAO, Jiangsu (CN); Jing QIAN, Shanghai (CN); Qi ZHOU, Shanghai (CN); Chenxi ZHANG, Beijing (CN); Yin QIAN, Shanghai (CN)

(21) Appl. No.: 18/696,634

(22) PCT Filed: Nov. 1, 2021
(86) PCT No.: PCT/CN2021/127937

§ 371 Date: Mar. 28, 2024

(51) Int. Cl. G16H 50/20; G16H 10/40; G16H 15/00; H04W 84/02

(52) U.S. Cl. CPC G16H 50/20; G16H 10/40; G16H 15/00; H04W 84/02

(57) ABSTRACT

A computer-implemented method is provided that includes transmitting, by a master node to a plurality of computing nodes, definition information about an initial medical validation model (410): performing, by the master node, a federated learning process together with the plurality of computing nodes (420), to jointly train the initial medical validation model using respective processed local training datasets available at the plurality of computing nodes, the respective local training datasets being processed by the plurality of computing nodes based on the definition information; and determining, by the master node, a final medical validation model based on a result of the federated learning process (430). Through the solution, by means of federated learning, it addresses the data security and privacy concerns from local sites owning.

### FIELD

Embodiments of the present disclosure generally relate to the field of computer science and in particular, to methods, devices, and computer program products for federated learning of a medical validation model.

### BACKGROUND

Medical tests are performed almost every day in medical laboratories and a large number of medical test reports are generated therefrom to present medical data. Before releasing the medical test reports to clinical departments or patients, validation procedures are initiated to ensure that the medical data presented in the reports are valid so as to avoid erroneous diagnoses on patients. However, lots of labor efforts are required in current validation procedures even though some automated functions have been introduced.

With the development of machine learning, currently it is proposed to train a machine learning model for automated validation of medical data. The training of the machine learning model may require training data including historical medical data. Conventionally, each lab or hospital may collect the historical medical data generated locally to train the machine learning model for use. However, such separate local training of models may be resource consuming and not very efficient. On the other hand, the available historical medical data may be limited at respective local sites. For example, most of the medical data collected at physical examination centers may reflect medical conditions of healthy people while most of the medical data collected at the oncology clinics may reflect medical conditions of tumor patents. Therefore, the medical validation model trained at one local site may not be generalized to provide accurate validation results for other local sites.

Therefore, it is desired to provide a solution for efficient and effective training of a medical validation model.

### SUMMARY

In general, example embodiments of the present disclosure provide a solution for federated learning of a medical validation model.

In a first aspect, there is provided a computer-implemented method. The method comprises transmitting, by a master node to a plurality of computing nodes, definition information about an initial medical validation model; performing, by the master node, a federated learning process together with the plurality of computing nodes, to jointly train the initial medical validation model using respective processed local training datasets available at the plurality of computing nodes, the respective local training datasets being processed by the plurality of computing nodes based on the definition information; and determining, by the master node, a final medical validation model based on a result of the federated learning process.

In a second aspect, there is provided a computer-implemented method. The method comprises receiving, by a computing node and from a master node, definition information about an initial medical validation model; processing a local training dataset at least based on the definition information; and performing a federated learning processing together with the master node and at least one further computing node, to jointly train the initial medical validation model using the processed local training dataset.

In a third aspect, there is provided an electronic device. The electronic device comprises at least one processor; and at least one memory comprising computer readable instructions which, when executed by the at least one processor of the electronic device, cause the electronic device to perform the steps of the method in the first aspect described above.

In a fourth aspect, there is provided an electronic device. The electronic device comprises at least one processor; and at least one memory comprising computer readable instructions which, when executed by the at least one processor of the electronic device, cause the electronic device to perform the steps of the method in the second aspect described above.

In a fifth aspect, there is provided a computer program product. The computer program product comprises instructions which, when executed by a processor of an apparatus, cause the apparatus to perform the steps of any one of the methods in the first aspect described above.

In a sixth aspect, there is provided a computer program product. The computer program product comprises instructions which, when executed by a processor of an apparatus, cause the apparatus to perform the steps of any one of the methods in the second aspect described above.

It is to be understood that the summary section is not intended to identify key or essential features of embodiments of the present disclosure, nor is it intended to be used to limit the scope of the present disclosure. Other features of the present disclosure will become easily comprehensible through the following description.

### BRIEF DESCRIPTION OF THE DRAWINGS

The following detailed description of the embodiments of the present disclosure can be best understood when read in conjunction with the following drawings, where:

FIG. 1 illustrates an example environment in which embodiments of the present disclosure may be implemented;

FIG. 2 illustrates a block diagram of a system for federated learning and application of a medical validation model according to some embodiments of the present disclosure;

FIG. 3 illustrates a block diagram of a computing node and a master node in the system of FIG. 2 for federated learning of a medical validation model according to some embodiments of the present disclosure;

FIG. 4 illustrates a flowchart of an example process for training of a medical validation model implemented at a master node according to some embodiments of the present disclosure;

FIG. 5 illustrates a flowchart of an example process for training of a medical validation model implemented at a computing node according to some embodiments of the present disclosure; and

FIG. 6 illustrates a block diagram of an example computing system/device suitable for implementing example embodiments of the present disclosure.

Throughout the drawings, the same or similar reference numerals represent the same or similar element.

### DETAILED DESCRIPTION

Principle of the present disclosure will now be described with reference to some embodiments. It is to be understood that these embodiments are described only for the purpose of illustration and help those skilled in the art to understand and implement the present disclosure, without suggesting any limitation as to the scope of the disclosure. The disclosure described herein can be implemented in various manners other than the ones described below.

In the following description and claims, unless defined otherwise, all technical and scientific terms used herein have the same meaning as commonly understood by one of ordinary skills in the art to which this disclosure belongs.

References in the present disclosure to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an example embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the listed terms.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof.

As used herein, the term “model” is referred to as an association between an input and an output learned from training data, and thus a corresponding output may be generated for a given input after the training. The generation of the model may be based on a machine learning technique. The machine learning techniques may also be referred to as artificial intelligence (AI) techniques. In general, a machine learning model can be built, which receives input information and makes predictions based on the input information. For example, a classification model may predict a class of the input information among a predetermined set of classes. As used herein, “model” may also be referred to as “machine learning model”, “learning model”, “machine learning network”, or “learning network,” which are used interchangeably herein.

Generally, machine learning may usually involve three stages, i.e., a training stage, a validation stage, and an application stage (also referred to as an inference stage). At the training stage, a given machine learning model may be trained iteratively using a great amount of training data until the model can obtain, from the training data, consistent inference similar to those that human intelligence can make. Through the training, the machine learning model may be regarded as being capable of learning the association between the input and the output (also referred to an input-output mapping) from the training data. The set of parameter values of the trained model is determined. At the validation stage, a validation input is applied to the trained machine learning model to test whether the model can provide a correct output, so as to determine the performance of the model. At the application stage, the resulting machine learning model may be used to process an actual model input based on the set of parameter values obtained from the training and to determine the corresponding model output.

### Example Environment

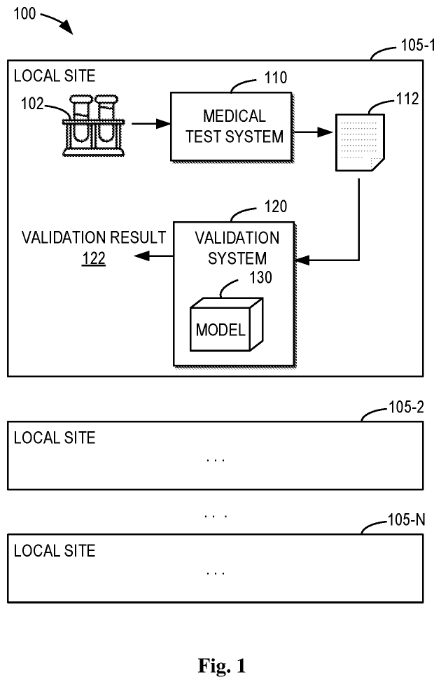

As mentioned above, validation procedures are important to ensure validity of medical data that are generated in various medical tests. FIG. 1 illustrates an environment 100 in which example embodiments of the present disclosure can be implemented. The environment 100 involves a typical workflow for medical diagnostic testing implemented at different local sites 105-1, 105-2, . . . 105-N, where N is larger than or equal to one. For the purpose of discussion, the local sites 105-1, 105-2 . . . , 105-N are collectively or individually referred to as local sites 105 hereinafter. The local sites 105 may include medical labs, hospitals, clinic departments, physical examination centers, medical institutions, or any sites where medical tests are carried out and medical data resulting from the medical tests are needed to be validated.

In FIG. 1, for the purpose of brevity, the workflow for medical diagnostic testing is detailed in the local site 105-1 although it would be appreciated that similar workflows are implemented at other local sites. The workflow generally includes performing a medical test on a test sample for medical diagnostics, generating medical data in the medical test, and validating the generated medical data.

As shown in FIG. 1, at the local site 105, a medical test system 110 is configured to perform a medical test on a test sample 102 and generate medical data 112 associated with the test sample 102. The medical test may include an in-vitro diagnostic test, such as a biochemical detection test or an immuno-detection test. The medical test system 110 may include one or more automated laboratory instruments or analytical apparatuses designed for analysis of test samples via various chemical, biological, physical, or other medical test procedures. In some examples, the instruments or analytical apparatuses can be configured to induce a reaction of a sample with a reagent for obtaining a measurement value. Examples of such instruments or analytical apparatuses are clinical chemistry analyzers, coagulation analyzers, immunochemistry analyzers, hematology analyzers, urine analyzers and nucleic acid analyzers that are used for the qualitative and/or quantitative detection of analytes present in the samples, to detect the result of chemical or biological reactions and/or to monitor the progress of chemical or biological reactions.

The medical test system 110 may be operable to perform a medical test to measure the parameters of the sample or at least one analyte thereof. The medical test may involve one or more test items conducted on the sample 102. The medical test system 110 may return test results corresponding to respective test items as the medical data 112. Possible test results returned by the medical test system 110 may be obtained by determining concentrations of the analyte in the sample, a digital (yes or no) result indicating existence of the analyte in the sample (corresponding to a concentration above the detection level), data obtained from mass spectroscopy of proteins or metabolites and physical, mechanical, optical, electrical or chemical parameters of various types, and/or the like.

Some specific examples of types of test items may include levels of alanine aminotransferase (ALT), aspartate aminotransferase (AST), glutamic dehydrogenase (GLDH), concentration of sodium (NA), age, hemoglobin, plasma protein, albumin (ALB), globulin (GLB), total bilirubin (TBIL), direct bilirubin (DBIL), total bile acid (TBA), blood urea nitrogen (BUN), and so on. The examples listed here are not exhaustive. The test items to be performed in a specific medical test may be specified by an entity who requests the medical test, such as a clinic department, a physical examination center, a doctor, a patient, or the like.

The test sample 102 may also be referred to as a biological sample, which is a biological material(s) suspected of containing one or more analytes of interest and whose detection, qualitative and/or quantitative may be associated to a clinical condition. The biological sample is derived from a biological source, such as a physiological fluid, including blood, saliva, ocular lens fluid, cerebrospinal fluid, sweat, urine, stool, semen, milk, ascites fluid, mucous, synovial fluid, peritoneal fluid, amniotic fluid, tissue, cells, or the like. Such biological source may be collected from a biological object, for example, a patient, a person, an animal, or the like.

The biological sample can be pretreated prior to use, such as preparing plasma or serum from blood. Methods of treatment can involve centrifugation, filtration, distillation, dilution, concentration and/or separation of sample components including analytes of interest, inactivation of interfering components, and addition of reagents. A biological sample may be used directly as derived from the source or used following a pretreatment to modify the character of the sample. In some embodiments, an initially solid or semi-solid biological material can be rendered liquid by dissolving or suspending it with a suitable liquid medium.

The term “reagent” refers to a substance which is added to a biological sample when performing a particular medical test on the biological sample to elicit a particular reaction in the sample. The reagents can be specific for a particular test or assay. For example, in a situation where a partial thromboplastic time of a blood sample shall be determined, the analyzer can be configured to add an activator as reagent to the blood sample to activate the intrinsic pathway of coagulation. Particular substances can be “modifying agents” or “reagents” in different situations. In some examples, a reagent may not be added to the biological sample to be tested.

The medical data 112 associated with the test sample 102 may include one or more test results of test items conducted in the medical test at the medical test system 110. The types of test results may be specified by an operator of the medical test system 110 (for example, a laboratory technician) or otherwise automatically identified from an electronic order via an information system connected with the medical test system 110. In some examples, the medical data 112 may be organized in a medical test report with specific test items and corresponding test results listed thereon. In some examples, in addition to the test results generated in the medical test, the medical data 112 may also include auxiliary information, such as information related to the test sample 102 and/or the biological object (such as the patient) from which the test sample 102 is collected.

The medical data 112 is provided to a validation system 120 to evaluate validity of the medical data 112 and determine whether the medical data 112 can be released or not. The need for validation is because many potential problems can occur during the sample gathering and testing processes. For example, a patient sample may be mislabeled, resulting in test results being reported in association with the wrong patient. As another example, the patient sample may have been improperly drawn or improperly handled, resulting in sample contamination and erroneous test results. Furthermore, a laboratory analyzer may be either malfunctioning or drifting out of calibration, again causing the analyzer to report erroneous results.

To improve the efficiency of the medical validation process, a trained medical validation model 130 may be utilized in the validation system 120, to automatically evaluate validity of the medical data 112. The medical validation model 130 is trained to automatically process the input medical data and output a validation result indicating one of the validation categories. The trained medical validation model 130 each represents an association between medical data and the validation categories.

The input to the medical validation model 130 is medical data, and an output validation result 122 from the medical validation model 130 comprises one of the validation categories. In some embodiments, the medical validation model 130 may be designed as a classification model for classifying/assigning the input medical data into one of the validation categories. In some embodiments, the validation result 122 from the medical validation model 130 may include an explicit indication of a validation category and/or a confidence level of the validation category for the current medical data. In determining the output validation category, the medical validation model 130 measures respective probabilities of the predetermined validation categories and select the one that has the highest probability.

The medical data may include one or more test results of test items, which may include measure values related to the test items and/or a digital (yes or no) result indicating existence of a certain analyte in the test sample. The medical data 112 may further include other information such as patient information, department information, and/or the like.

Each of the validation categories output by the medical validation model may indicate one of predetermined actions to be performed on the medical data, which can be considered as a suggestion for the system or a user to automatically or manually decide how the medical data can be treated in a next step of the whole medical diagnostic testing workflow.

The medical validation is to find potential errors in the medical data before the medical data is released to an entity who requests the medical test (such as the clinical department or the patient). If the medical data is validated as correct and having no error, the next step is to release the medical data to that entity (or to require a quick manual review and then release to the entity). In this case, one possible action to be performed on the medical data is to release the medical data to an entity who requests the medical test related to the medical data directly or after a quick manual review. The validation result 122 may include a validation category indicating that the medical data 112 is correct to be directly released (or released after a simple manual review) to a requestor who orders the medical test.

In other cases, the medical data is validated as having an error due to the test sample, the performed medical diagnostic testing procedures, the reagent used in the medical test, mismatching with the physical condition of the biological object of the test sample, insufficient information for decision making, or the like. In such cases, corresponding actions are needed to be performed to correct the error. The action indicated in a validation result 122 for medical data is to suggest further validation of the medical data. This action is a general suggestion, which means that the current medical data should not be released and a manual review is required to decide how the medical data can be further validated.

In some examples, one or more specific actions for further validation may be indicated by validation categories output from the medical validation model 130, including an action of re-running the medical test related to the medical data; an action of checking a historical patient medical record; an action of checking reaction of a reagent in the medical test, such as checking a reagent reacting curve; an action of checking a test sample collected for use in the medical test; an action of checking the medical data in combination with clinical diagnosis; and an action of checking patient drug use; and/or the like. It would be appreciated that the next-step actions listed above are merely some specific examples, and more, less, or different actions can also be specified as required in actual use cases and accordingly the validation categories for the medical validation model 130.

### Working Principle and Example System

The introduction of the medical validation model can significantly reduce manual efforts paid in reviewing the medical data and also can improve accuracy and quality in medical data validation. However, due to various reasons, currently each local site (e.g., a lab or hospital) trains its own medical validation model using medical data that are collected locally, which may be resource consuming and not very efficient. On the other hand, the medical validation model trained at a local site may not be generalized to provide accurate validation results for other local sites.

A straightforward solution is to collect historical medical data from different local sites to train a model at a center node. However, it is not practical and ideal considering the sensitivity of medical data and the poor networking connections among different local sites. Some local sites may refuse to export their medical data due to some agreements or regulations.

According to example embodiments of the present disclosure, there is proposed a solution for federated learning of medical validation. In this solution, a master node and a plurality of computing nodes work together to perform a federated learning process, to jointly train a medical validation model. The master node provides the computing nodes with definition information about the medical validation model. The computing nodes process local training datasets respectively based on the definition information and utilize the processed local training datasets in the federated learning process. As such, a local training dataset itself at a computing node may not be exposed to the master node or other computing nodes. The master node determines a final medical validation model based on a result of the federated learning process.

Through the solution, by means of federated learning, it addresses the data security and privacy concerns from local sites owning the training datasets for model training. In addition, the final medical validation model has been trained with different local training datasets, which enables improved accuracy and quality in medical validation using the model.

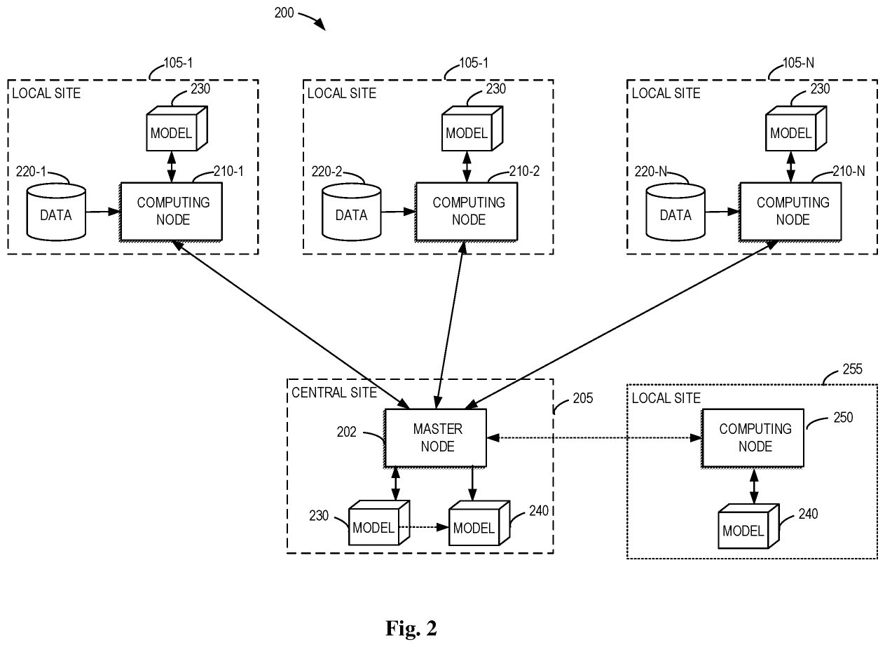

In the following, example embodiments of the present disclosure are described with reference to the drawings. Reference is first made to FIG. 2, which illustrates a system 200 for federated learning and application of a medical validation model. The system 200 in FIG. 2 may be partially implemented in the environment 100 in FIG. 1. For the purpose of discussion, reference is made to FIG. 1 to describe the system 200.

As illustrated, the system 200 includes a master node 202 and a plurality of computing nodes 210-1, 210-2, . . . , 210-N (collectively or individually referred to as computing nodes 210 hereinafter). The master node 202 and the computing nodes 210 may comprise or implement as any number of devices/systems having computing capabilities, such as servers, computers, mainframes, and the like.

The computing nodes 210-1, 210-2, . . . , 210-N may be each deployed at the local sites 105-1, 105-2, . . . 105-N or can otherwise access to data available at the local sites. Thus, the computing nodes 210-1, 210-2 . . . 210-N may be considered as local nodes to those sites. As illustrated in FIG. 2, the computing node 210-1 can access to data stored in a database 220-1 which are available at the local site 105-1, the computing node 210-2 can access to data stored in a database 220-2 which are available at the local site 105-2, the computing node 210-N can access to data stored in a database 220-N which are available at the local site 105-N, and so on. For the purpose of discussion, the databases 220-1, 220-2 . . . 220-N are collectively or individually referred to as databases 220 hereinafter.

As will be discussed in detail below, according to embodiments of the present disclosure, the master node 202 and the plurality of computing nodes 210 work together to jointly train an initial medical validation model 230 by means of federated learning. The computing nodes 210 obtain respective local training datasets for the federated learning from their accessible databases 220. In such a case, the local sites 105 may be referred to as contribution sites because they contribute their data for global model training. The computing nodes 210 process the local training datasets based on definition of the initial medical validation model 230 received from the master node 202. The definition information may define one or more aspects of the input and output of the initial medical validation model 230 to be trained. With the definition information, the local training datasets may be adapted at the computing nodes to be suitable for training a global model. The master node 202 can determine a final medical validation model 240 based on a result of the federated learning.

Federated learning is a machine learning technique that trains a model across multiple decentralized nodes holding the training datasets. The federated learning enables the computing nodes 210 to collaboratively learn a model while keeping all the training datasets on nodes. That is, the local training datasets are not exposed to other computing nodes 210 or the master node 202 during the training. As a result, the local sites 105 have no privacy concerns and data ownership concerns since the raw medical data never leaves the local computing nodes 210. In addition, the security concerns are greatly reduced because there is no single node at which a security breach can compromise a large body of raw data.

The master node 202 and the computing nodes 210 may be deployed with respective federated learning engines to implement a federated learning process for the initial medical validation model 230. There are various federated learning frameworks that can be applied in the embodiments of the present disclosure. As some examples, the applicable federated learning frameworks may include Tensorflow Federated (TFF), Pysyft, or Federated AI Technology Enabler (FATE), and any other federated learning frameworks that are currently available or to be developed in the future.

To support the federated learning among the nodes, in the system 200, the master node 202 is communicatively connected with the plurality of computing nodes 210. In some embodiments, a star topology network may be established among the master node 202 and the computing nodes 210. In some embodiments, in the star topology network, outbound connections from the respective computing nodes 210 to the master node 202 are allowed, but inbound requests to the respective computing nodes 210 are not allowed. The outbound connections can further ensure data security at the computing nodes 210.

In some embodiments, the resulting final medical validation model 240 may be distributed to local sites for use in medical validation. The local site which receives the final medical validation model 240 for use may be referred to as a consumer site. In some embodiments, the final medical validation model 240 may be distributed to one or more sites other than the local sites 105 which serve as contribution sites, such as a local site 255 as illustrated in FIG. 2. For example, the master node 202 may distribute the final medical validation model 240 to a computing node 250 at the local site 255. In some embodiments, the master node 202 may alternatively or additionally distribute the final medical validation model 240 to one or more of the local sites 105 which contribute the training data for model training.

### Federated Learning of Medical Validation Model

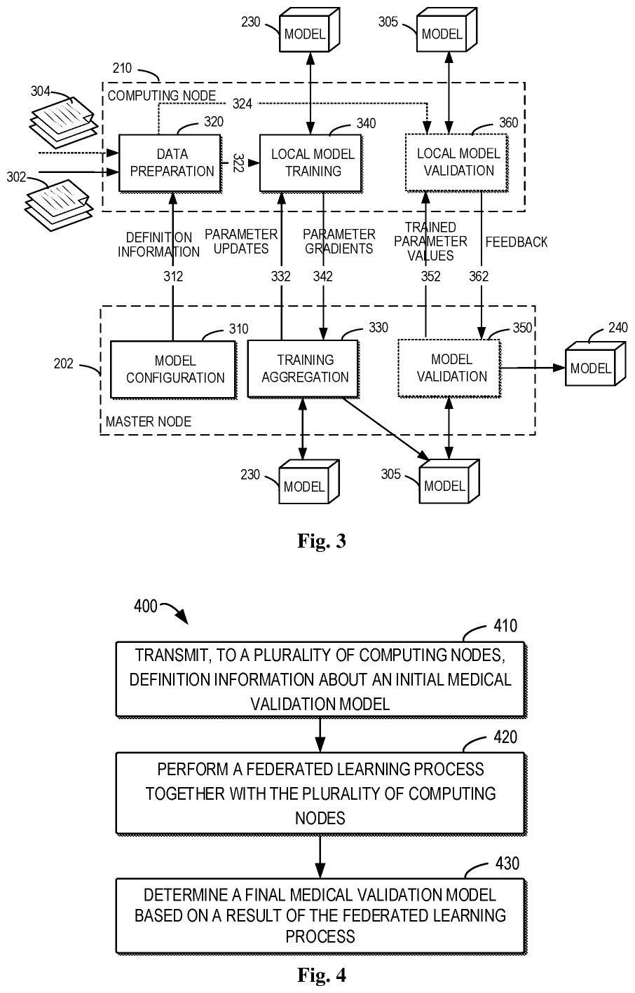

FIG. 3 illustrates a block diagram of a computing node 210 and a master node 202 in the system 200 of FIG. 2 for federated learning of a medical validation model according to some embodiments of the present disclosure. In FIG. 3, for the purpose of brevity, the example details structure of one computing node 210 and interaction between the master node 202 and this computing node 210 are illustrated. It is noted that each of the computing nodes 210 involved in the federated learning may include the same or similar components as illustrated in the computing node 210 in FIG. 3.

As illustrated, the master node 202 comprises a model configuration module 310 to configure an initial medical validation model 230 to be trained to the plurality of computing nodes, and a training aggregation module 330 to perform a federated learning process with the plurality of computing nodes 210 and aggregate intermediate training results during the federated learning process. A computing node 210 comprises a data preparation module 320 to pre-process data from the database 220 which at least partially forms a training dataset for model training, and a local model training module 340 to perform the federated learning process based on the training dataset prepared by the data preparation module 320.

In some embodiments, the master node 202 and the plurality of computing nodes 210 may implement a validation stage for machine learning, to evaluate performance of a trained medical validation model 305 determined from the federated learning process so as to determine a final medical validation model 240 for distribution. The master node 220 may include a model validation module 350 and the computing node 210 may include a local model validation module 360 to implement the validation stage of the trained medical validation model 305.

The modules in the master node 202 and the computing nodes 210 may be implemented as one or more software engines, hardware components, middleware components, and/or the like, which are configured with logic for implementing the functionalities attributed to the particular modules.

Detailed description of the respective modules in the master node 202 and the computing nodes 210 will be provided below.

The model configuration module 310 of the master node 202 is configured to transmit definition information 312 about an initial medical validation model 230 to the computing nodes 210, e.g., the data preparation module 320 in a computing node 210. The definition information is used to define the initial medical validation model 230 globally among the computing nodes 210. The initial medical validation model 230 may be defined similarly as the medical validation model 130 as described with reference to FIG. 1. As used herein, an “initial” medical validation model indicates that the medical validation model has initial parameter values which may be updated iteratively during the training process.

In some embodiments, the definition information 312 may define one or more aspects of the input and output of the initial medical validation model 230 to be trained. In some embodiments, the definition information 312 may further define a model construction of the initial medical validation model 230, including the model type, layers, processing units in the layers, connections between the processing units in the initial medical validation model 230.

The data preparation module 320 in each computing node 210 is configured to obtain a local training dataset 302 from the database 220 and process the local training dataset 302 based on the definition information 312 to obtain a processed local training dataset 322 to provide to the local model training module 340.

Since the initial medical validation model 230 is defined by the master node 202 globally among different local sites 105, the local training datasets available at the local sites 105 may be not suitable for training the initial medical validation model 230. In addition to define the model configuration, the definition information 312 from the master node 202 may at least allow the computing nodes 210 to prepare the local training datasets to be ready for training the initial medical validation model 230.

As mentioned above, the input to the initial medical validation model 230 may include medical data and the output (i.e., a validation result) from the initial medical validation model 230 may indicate one of a plurality of validation categories which correspond to predetermined actions to be performed on the input medical data.

In some embodiments, the medical validation model 130 may be locally trained in a supervised manner. Thus, the local training dataset 302 at a local site 105 may include historical medical data generated in medical tests and labeling information associated therewith. The historical medical data may include a number of medical test reports that are generated in different medical tests for one or more patients.

A medical test report may indicate item names and corresponding item values, including test item names and corresponding test values, item names indicating auxiliary information such as information related to the test sample 102 and/or the biological object (such as the patient) from which the test sample 102 is collected. The labeling information indicates respective local validation categories corresponding to the historical medical data. The labeling information may be used as ground-truth validation categories in the training. Generally, the labeled local validation categories at each local site 105 may indicate the actions that were considered to be the right actions for the historical medical data and/or those that are marked manually by the laboratory experts.

In some cases, in the local training datasets 302, different local sites 105 may utilize different item names to identify the same items included in the historical medical data. For example, a local site 105 may record a test item with an item name “Serum total prostate-specific antigen” while other local sites 105 may record the same test item with an item code “tPSA” or “PSA.” To avoid the medical validation model to treat the same item with different item names as different items, in some embodiments, the model configuration module 310 in the master node 202 may determine the definition information 312 to indicate unified item names in medical data input to the initial medical validation model 230. According to the definition information 312, the data preparation module 320 in a commuting node 210 may map local item names used in the historical medical data of the local training dataset 302 to the unified item names. That is, the data preparation module 320 may identify a local item name that identifies the same item as a unified item name indicated by the definition information 312, and replace the local item name in the historical medical data with the corresponding unified item name if the local item name is different from the unified item name.

Table 1 shows an example of mapping between unified item names and local item names.

TABLE 1
Mapping between unified item names and local item names

| | Local site 105-1 | | Local site 105-2 | | Local site 105-N | |
|---|---|---|---|---|---|---|
| Unified | | Local item | | Local item | | Local item |
| item names | Has? | name | Has? | name | Has? | name |
| Gender | Y | Gender | Y | Gender | Y | Gender |
| Age | Y | Age | Y | Age | Y | Age |
| Department1 | Y | Department1 | Y | Department1 | Y | Department1 |
| TestItem1 | Y | TestItem1 | | | | |
| TestItem2 | Y | TestItem2 | Y | TestItem2 | | |
| TestItem3 | Y | TestItem3 | Y | TestItem3 | | |
| TestItem4 | | | | | Y | TestCode1 |
| TestItem5 | | | Y | TestItem5 | Y | TestCode2 |
| TestItem . . . | | | Y | TestItem . . . | Y | TestCode3 |
| TestItemn | Y | TestItemn | | | Y | TestCode4 |

In the example of Table 1, local item names in the historical medical data available at the local sites 105-1 and 105-2 have the same local item names as the unified test item names. For the local site 105-N, the local item names “TestCode1,” “TestCode2,” “TestCode3,” and “TestCode4” each refers to the same items as the unified item name “TestItem4,” “TestItem5,” “TestItem . . . ” and “TestItemn” indicated in the definition information. The computing node 210 at the local site 105-N may update the local training dataset 302 by replacing the local item names with the unified item names.

In some embodiments, the definition information 312 may indicate unified item names of all possible items included in an input to the initial medical validation model 230. If historical medical data in a local training dataset 302 available at a local site 105 includes no such local items or local item names, the data preparation module 320 may also be able to include the missing items with the unified item names in an input to the initial medical validation model 230.

In some embodiments, at a local site 105, if an item is indicated by the definition information 312 as forming the input to the initial medical validation model 230 but a value of the indicated item is unavailable from historical medical data in a local training dataset 302, the corresponding computing node 210 may need to process the historical medical data to be suitable for the initial medical validation model 230. As the initial medical validation model 230 is trained globally, the input may generally include a large number of items that are considered to be relevant with the validation categories.

The medical data obtained at a local site 105 may not include all the items, which may result in a sparse matrix issue and in turn leads to low accuracy of the resulting model. For example, for a same medical test, some local sites 105 may record values of five test items while other local sites 105 may record values of ten test items. The input to the initial medical validation model 230 may indicate more test items than some of the local sites. It can also be seen from Table 1, test items “TestItem4,” “TestItem5,” and “TestItem . . . ” are missing from the local site 105-1 while the test items “TestItem4” and “TestItemn” are missing from the local site 105-2.

In some embodiments, to address the sparse matrix issue, a computing node 210 (i.e., the data preparation module 320 therein) may process its local training dataset 302 by filling in a predetermined value(s) for an item(s) that is unavailable in the local historical medical data but is required to be included in the input to the initial medical validation model 230.

The predetermined value for a certain item may be determined in various ways. In some embodiments, the predetermined value may be determined as an average value of a reference value range of the indicated item. The reference value range is used to identify a normal situation for the indicated item, and any value lower than the lower limit or higher than the upper limit of the reference value range may be considered as an outlier value. The use of the average value of the reference value range may not affect the validation result of the medical data in which the indicated item is included. In some embodiments, the predetermined value may be determined as a median value of available values of the indicated item in historical medical data generated in other medical tests. For example, among all the historical medical data generated multiple medical tests, the value of the indicated item may be missing from one or some of the medical tests. In such a case, other available values of the same item may be used to determine the predetermined value to be filled in.

In some embodiments, the predetermined value for a certain item may be determined as in many other ways, such as a fixed value configured by the master node 202. In some embodiments, instead of filling in predetermined values for the missing items, the computing nodes 210 may process the historical medical data by marking the missing items with untested. In some embodiments, the computing nodes 210 (i.e., the data preparation module 320) may address the sparse matrix issue by marking the values of the missing items as an extreme value (e.g., −9999) and transforming a sparse matrix constructed from the input historical medical data into a dense matrix. The computing nodes 210 may apply any suitable approaches for transformation from a sparse matrix to a dense matrix, one example approach of which may include Principal Component Analysis.

In some embodiments, some items in the medical data input to the initial medical validation model 230 may have numeric values. Sometimes different local sites 105 may record values of the same item with different units, leading to a data scaling problem. To deal with the potential data scaling problem among different local sites 105, the model configuration module 310 in the master node 202 may determine the definition information 312 to indicate a scaled value range for an item in medical data input to the initial medical validation model 230. The scaled value range may be, for example, a range from zero to one, or any other range. With the scaled value range for a certain item, the computing nodes 210 (i.e., the data preparation module 320) may process the historical medical data in the local training dataset 302 by values of this item in the historical medical data values within the scaled value range. As such, values from a same range (i.e., the scaled value range) may be determined for the same item across different local sites 105, which facilitates the feature engineering in the initial medical validation model 230. This may be practical because the federated learning assumes that the same feature of the input may follow the same distribution across various local sites.

In some embodiments, for an item in the medical data, its value may be calculated from two or more values of other items under test. For example, the ratio of free prostate specific antigen (FPSA) to total prostate specific antigen (TPSA), FPSA/TPSA is calculated from the values of FPSA and TPSA, and the anion gap is calculated based on the difference between primary measured cations (sodium Na+ and potassium K+) and the primary measured anions (chloride Cl− and bicarbonate HCO3−) in serum. The computing nodes 210 (i.e., the data preparation module 320) may locally calculate the value of the item based on the original values of the other items before scaling them into the scaled value range.

In some cases, other than the input item names, different local sites 105 may apply different criteria to divide the historical medical data into different sets of validation categories. For example, a local site 105 may label historical medical data in the local training dataset 302 with two validation categories, one indicating that the historical medical data is correct to be directly released and the other one indicating the further validation is needed. Another local site 105 may label historical medical data with more than two validation categories indicating specific actions to be subjected to further validation. To allow the local model training module 340 to perform the training in a supervised manner, in some embodiments, the model configuration module 310 in the master node 202 may determine the definition information 312 to indicate unified validation categories output from the initial medical validation model 230.

According to the definition information 312, the data preparation module 320 in a commuting node 210 may map the local validation categories to the unified validation categories. That is, the data preparation module 320 may apply the same labeling approach in updating or creating the labeling information in the local training dataset 302. In some examples, the data preparation module 320 may preserve the local validation categories that are the same as the unified validation categories (for example, those with the same category names labeled on the historical medical data in the local training dataset 302). In some examples, if historical medical data in the local training dataset 302 is labeled with a local validation category but the definition information 312 indicates that this local validation category is divided in a fine-grained way and mapped to two or more unified validation categories, the data preparation module 320 may divide the historical medical data and label them with the two or more corresponding unified validation categories. In some other examples, historical medical data labeled with two or more local validation categories in the local training dataset 302 may be aggregated and labeled with one unified validation category to which the two or more local validation categories are mapped.

In some embodiments, the definition information may further indicate one or more unified red flag rules for medical data prevented from being input to the initial medical validation model. As the medical validation procedure is to make sure that medical test reports with potential errors are not released, a red flag rule may be set to make sure that the medical test reports with significant or obvious errors are not accidently determined as being correct by a medical validation model, considering potential wrong diagnosis performed by the model. More specifically, medical data satisfying a red flag rule may be directly passed to manual validation, instead of being input to a medical validation model for automated validation. Depending on different requirements and different regulations to be followed, different local sites 105 may apply different local red flag rules to block medical test reports satisfying the local red flag rules from being passed to the model-based automated validation.

In training the initial medical validation model 230, the master node 202 may configure one or more unified red flag rules in the definition information 312, to allow the computing nodes 210 to apply unified data filtering for medical data that can be input to the initial medical validation model 230. A computing node 210 (e.g., the data preparation module 320 in the computing node 210) may process the local training dataset 302 by filtering out historical medical data satisfying the one or more unified red flag rules. A unified red flag rule may define a threshold-based criterion for an item in a medical test report. For example, a unified red flag may define that any medical test report with serum potassium higher than a threshold may not be released. If the value of this item in a historical medical test report satisfies the threshold-based criterion, for example, if the value exceeds the threshold or is below the threshold (depending on how the criterion is set), the historical medical test report may be excluded from the local training dataset 302.

By applying the unified red flag rule(s) to filter the local training datasets, medical data satisfying the unified red flag rule(s) may not be used to train the initial medical validation model 230, which means that the model may probably not learn knowledge from the medical data satisfying the unified red flag rule(s). At the following application stage when the final medical validation model 240 is applied, medical data at the consumer sites may also be filtered with the same unified red flag rule(s) in order to guarantee the validation accuracy.

In some embodiments, as an alternative, the master node 202 may not configure a unified red flag rule for the local training datasets at the local sites 105. As such, the initial medical validation model 230 may be trained without any limitation on the training data selection. As a result, the final medical validation model 240 is a rule-free model. At the following application stage of the final medical validation model 240, the consumer sites may apply respective local red flag rule(s) to determine which medical data can be passed to the final medical validation model 240 for automated validation.

Some example embodiments of model configuration and data preparation have been described above. After the data preparation, each computing node 210 may generate a processed local training dataset 322 for training. As indicated, the master node 202 works together with the computing nodes 210 at the local sites 105 to perform a federated learning process, so as to jointly train the initial medical validation model 230. During the federated learning, the local model training module 340 in a computing node 210 may train the local medical validation model 230 locally using the processed local training dataset 322. The computing node 210 may apply a corresponding training algorithm to perform the training.

In some embodiments, the computing node 210 may generate parameter gradients 342 based on the processed local training dataset 322 and transmit the parameter gradients 342 to the training aggregation module 330 in the master node 202. The training aggregation module 330 may aggregate the parameter gradients received from the plurality of computing nodes 210 to determine parameter updates 332 to the parameters of the initial validation model 230. The parameter updates 332 may be transmitted to the plurality of computing nodes 210. In some embodiments, the parameter gradients 342 and/or the parameter updates 332 may be communicated in a secure channel between the computing nodes 210 and the master node 202 to prevent from information leakage.

With the parameter updates 332, the local model training module 340 in a computing node 210 may determine updated parameter values for the initial validation model 230, to form an intermediate initial validation model and perform further training steps on the basis of the intermediate initial validation model using the processed local training dataset 322. The exchange of parameter gradients and parameter updates between the master node 202 and the computing nodes 210 may be iteratively performed until a convergence condition for the federated learning process is reached. At this time, the training aggregation module 330 in the master node 202 may obtain the trained medical validation model 305 with trained parameter values determined from the federated learning process.

In some embodiments, the master node 202 may determine the trained medical validation model 305 as the final medical validation model 240 that is ready to be distributed to the consumer sites. In some embodiments, the master node 202 may perform a model validation procedure to validate if the performance of the trained medical validation model 305 is good to be distributed. Since the master node 202 may not have data to validate the model and considering that different local sites 105 may have different validation criteria, the master node 202 may work with the computing nodes 210 at the local sites 105 to perform the model validation procedure.

Specifically, the model validation module 350 in the master node 202 may distribute the trained medical validation model 305 to the plurality of computing nodes 210, for example, by transmitting the trained parameter values 352 of the trained medical validation model 305 to the computing nodes 210. The local model validation module 360 in a computing node 210 may determine a performance metric of the trained medical validation model 305 using a processed local validation dataset 324. The processed local validation dataset 324 may be determined from an original local validation dataset 304 obtained from the database 220 in the corresponding local site 105. The processing of the local validation dataset 304 may be similar to the processing of the local training dataset 302 and the definition information 312 may also be utilized for the processing. During the local model validation process, the local model validation module 360 in a computing node 210 may input historical medical data in the processed local validation dataset 324 to the trained medical validation model 305 and determine whether the predicted validation result (indicating a validation category) output from the trained medical validation model 305 matches the ground-truth validation result in the processed local validation dataset 324.

Depending on the result of the local model validation process, the local model validation module 360 in the computing node 210 may determine a performance metric to indicate the performance of the trained medical validation model 305. The performance metric may, for example, indicate the precision rate or a loss rate of the predicted validation results output from the trained medical validation model 305. Alternatively or in addition, the performance metric may be determined based on a receiver operating characteristic (ROC) curve and/or an area under the curve (AUC). Other performance metrics may also be determined and the scope of the present disclosure is not limited in this regard. The local model validation module 360 in each computing node 210 may transmit the performance metric as a feedback 362 to the model validation module 350 in the master node 202.

After gathering the feedback of the performance metrics of the trained medical validation model 305 from the plurality of computing nodes 210, the model validation module 350 in the master node 202 may determine the final medical validation model 240 based on the received feedback. In some embodiments, if the received performance metrics meet a model release criterion, for example, the performance metrics from most or a certain number of the computing nodes 210 indicate that the trained medical validation model 305 works well in local medical validation, the model validation module 350 may determine that the trained medical validation model 305 may be distributed as the final medical validation model 240. In some embodiments, if the received performance metrics fail to meet the model release criterion, for example, the performance metrics from most or a certain number of the computing nodes 210 indicate that the trained medical validation model 305 have unsatisfied performance when operating locally, the model validation module 350 may determine that the trained medical validation model 305 may be further adjusted and thus a model fine-tuning process may be initiated, to further update the parameter values of the trained medical validation model 305.

In some embodiments, the model validation module 350 may distribute the trained medical validation model 305 as a final medical validation model to computing nodes 210 from which the satisfied performance metrics (such as those exceeding or equal to a performance threshold) are received. The model validation module 350 may distribute the trained medical validation model 305 to other local sites to request them to fine-tune the trained medical validation model 305 using their local training datasets.

In the above embodiments, the training and validation of one global medical validation model is described. In other embodiments, the master node 202 and the computing nodes 210 may jointly train a plurality of different medical validation models based on federated learning processes. The different medical validation models may be constructed with different processing algorithms (e.g., a model based on a logistic regression and a model based on a neural network), trained with different training algorithms, and so on. As such, the trained medical validation models from the federated learning processes may have varied performance even though they are trained with the same local training datasets at the computing nodes 210.

By obtaining different trained medical validation models and obtaining feedback indicating their performance metrics from the computing nodes 210, the model validation module 350 in the master node 202 may select one or more candidate medical validation models that have satisfied performance metrics for a certain consumer site (including the local sites 105 and other local sites such as the local site 255). The computing node at the consumer site may apply a local dataset to further validate the performance of the candidate medical validation models and select, based on performance metrics of the candidate medical validation models, an appropriate model for use in local medical validation. The computing node at the consumer site may fine-tune the candidate medical validation models using a local dataset if needed.

### Example Processes

FIG. 4 illustrates a flowchart of an example process for training of a medical validation model implemented at a master node according to some embodiments of the present disclosure. The process 400 can be implemented at the master node 202 in FIG. 2. For the purpose of discussion, the process 400 will be described with reference to FIG. 2.

At block 410, the master node 202 transmits, to a plurality of computing nodes 210, definition information about an initial medical validation model. At block 420, the master node 202 performs a federated learning process together with the plurality of computing nodes 210, to jointly train the initial medical validation model using respective processed local training datasets available at the plurality of computing nodes 210. The respective local training datasets are processed by the plurality of computing nodes 210 based on the definition information. At block 430, the master node 202 determines a final medical validation model based on a result of the federated learning process.

In some embodiments, the master node 202 may distribute the final medical validation model to at least one of the plurality of computing nodes 210 or at least one further computing node for use in medical validation.

In some embodiments, the respective local training datasets may comprise historical medical data generated in medical tests and labeling information indicating local validation categories of the historical medical data.

In some embodiments, the definition information indicates unified item names in medical data input to the initial medical validation model, and unified validation categories output from the initial medical validation model, the unified validation categories indicating a plurality of predetermined validation actions to be performed on the medical data. In some embodiments, the respective local training datasets may be processed by mapping local item names used in the historical medical data to the unified item names, and mapping the local validation categories to the unified validation categories.

In some embodiments, the definition information may further indicate a scaled value range for an item in medical data input to the initial medical validation model. In some embodiments, the respective local training datasets may be processed by mapping values of the item in the historical medical data into values within the scaled value range.

In some embodiments, the definition information may further indicate a unified red flag rule for medical data prevented from being input to the initial medical validation model. In such as case, the respective local training datasets may be processed by filtering out historical medical data satisfying the unified red flag rule.

In some embodiments, the definition information may indicate an item in medical data input to the initial medical validation model, and a value of the indicated item may be unavailable from historical medical data in a local training dataset. In such as case, the local training dataset are processed by filling in a predetermined value for the indicated item. In some embodiments, the predetermined value comprises either one of an average value of a reference value range of the indicated item and a median value of available values of the indicated item in historical medical data generated in other medical tests.

In some embodiments, to determine the final medical validation model, the master node 202 may obtain a trained medical validation model from the result of the federated learning process, and distribute the trained medical validation model to the plurality of computing nodes 210. The master node 202 may receive feedback from the plurality of computing nodes 210, which indicate respective performance metrics of the trained medical validation model determined by the computing nodes 210 using respective local validation datasets. The master node 202 may then determine the final medical validation model based on the received feedback. In some embodiments, in response to the respective performance metrics meeting a model release criterion, the master node 202 may determine the trained medical validation model as the final medical validation model. In some embodiments, in response to the respective performance metrics failing to meet the model release criterion, the master node 202 may adjust the trained medical validation model to generate the final medical validation model.

In some embodiments, the master node 202 is communicatively connected with the plurality of computing nodes 210 in a star topology network.

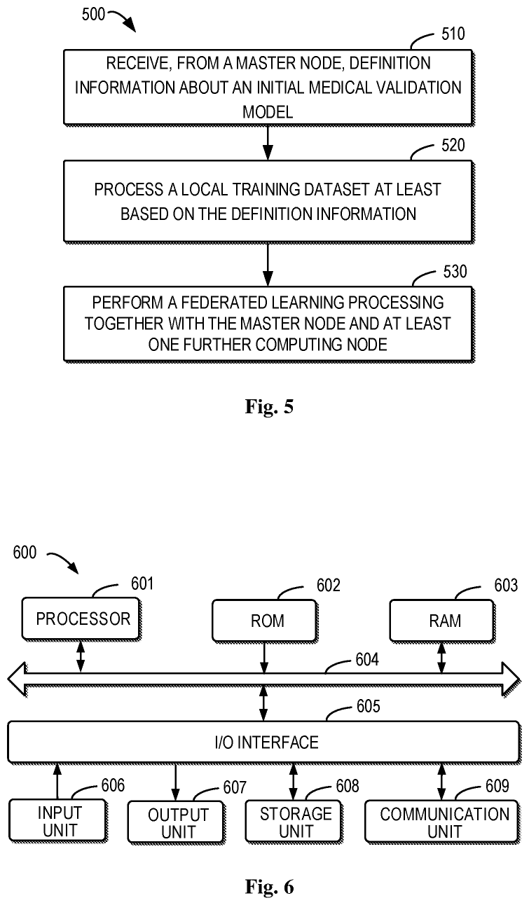

FIG. 5 illustrates a flowchart of an example process 500 for training of a medical validation model implemented at a computing node according to some embodiments of the present disclosure. The process 500 can be implemented at the computing node 210 in FIG. 2. For the purpose of discussion, the process 500 will be described with reference to FIG. 2.

At block 510, the computing node 210 receives from a master node 202 definition information about an initial medical validation model. At block 520, the computing node 210 processes a local training dataset at least based on the definition information. At block 530, the computing node 210 performs a federated learning processing together with the master node 202 and at least one further computing node, to jointly train the initial medical validation model using the processed local training dataset.

In some embodiments, the computing node 210 may further receive from the master node 202 a final medical validation model determined from the federated learning process.

In some embodiments, the local training dataset comprises historical medical data generated in medical tests and labeling information indicating local validation categories of the historical medical data.

In some embodiments, the definition information may indicate unified item names in medical data input to the initial medical validation model, and unified validation categories output from the initial medical validation model, the unified validation categories indicating a plurality of predetermined validation actions to be performed on the medical data. In some embodiments, the computing node 210 may map local item names used in the historical medical data to the unified item names, and mapping the local validation categories to the unified validation categories.

In some embodiments, the definition information may further indicate a scaled value range for an item in medical data input to the initial medical validation model. In some embodiments, the computing node 210 may map values of the item in the historical medical data into values within the scaled value range.

In some embodiments, the definition information may further indicate a unified red flag rule for medical data prevented from being input to the initial medical validation model. In some embodiments, the computing node 210 may filter historical medical data satisfying the unified red flag rule out from the local training dataset.

In some embodiments, the definition information may indicate an item in medical data input to the initial medical validation model, and a value of the indicated item is unavailable from historical medical data generated in a medical test. In some embodiments, the computing node 210 may process the historical medical data by filling in a predetermined value for the indicated item.

In some embodiments, the predetermined value may comprise either one of an average value of a reference value range of the indicated item and a median value of available values of the indicated item in historical medical data generated in other medical tests.

In some embodiments, the computing node 210 may further receive from the master node 202 a trained medical validation model determined from a result of the federated learning process. The computing node 210 may determine a performance metric of the trained medical validation model using a local validation datasets, and transmit to the master node 202 feedback indicating the determined performance metric.

In some embodiments, the computing node 210 may process a local validation dataset based on the definition information, and determine the performance metric using the processed local validation dataset.

### Example System/Device

FIG. 6 illustrates a block diagram of an example computing system/device 600 suitable for implementing example embodiments of the present disclosure. The system/device 600 can be implemented as or implemented in the master node 202 or the computing node 210 of FIG. 2. The system/device 600 may be a general-purpose computer, a physical computing device, or a portable electronic device, or may be practiced in distributed cloud computing environments where tasks are performed by remote processing devices that are linked through a communication network. The system/device 600 can be used to implement the process 400 of FIG. 4 and/or the process 500 of FIG. 5.

As depicted, the system/device 600 includes a processor 601 which is capable of performing various processes according to a program stored in a read only memory (ROM) 602 or a program loaded from a storage unit 608 to a random access memory (RAM) 603. In the RAM 603, data required when the processor 601 performs the various processes or the like is also stored as required. The processor 601, the ROM 602 and the RAM 603 are connected to one another via a bus 604. An input/output (I/O) interface 605 is also connected to the bus 604.

The processor 601 may be of any type suitable to the local technical network and may include one or more of the following: general purpose computers, special purpose computers, microprocessors, digital signal processors (DSPs), graphic processing unit (GPU), co-processors, and processors based on multicore processor architecture, as non-limiting examples. The system/device 600 may have multiple processors, such as an application-specific integrated circuit chip that is slaved in time to a clock which synchronizes the main processor.

A plurality of components in the system/device 600 are connected to the I/O interface 605, including an input unit 606, such as keyboard, a mouse, or the like; an output unit 607 including a display such as a cathode ray tube (CRT), a liquid crystal display (LCD), or the like, and a loudspeaker or the like; the storage unit 608, such as disk and optical disk, and the like; and a communication unit 609, such as a network card, a modem, a wireless transceiver, or the like. The communication unit 609 allows the system/device 600 to exchange information/data with other devices via a communication network, such as the Internet, various telecommunication networks, and/or the like.

The methods and processes described above, such as the process 400 and/or process 500, can also be performed by the processor 601. In some embodiments, the process 400 and/or process 500 can be implemented as a computer software program or a computer program product tangibly included in the computer readable medium, e.g., storage unit 608. In some embodiments, the computer program can be partially or fully loaded and/or embodied to the system/device 600 via ROM 602 and/or communication unit 609. The computer program includes computer executable instructions that are executed by the associated processor 601. When the computer program is loaded to RAM 603 and executed by the processor 601, one or more acts of the process 400 and/or process 500 described above can be implemented. Alternatively, processor 601 can be configured via any other suitable manners (e.g., by means of firmware) to execute the process 400 and/or process 500 in other embodiments.

### Enumerated Example Embodiments

The embodiments of the present disclosure may be embodied in any of the forms described herein. For example, the following enumerated example embodiments describe some structures, features, and functionalities of some aspects of the present disclosure disclosed herein.

In a first aspect, example embodiments of the present disclosure provide a computer-implemented method. The method comprises transmitting, by a master node to a plurality of computing nodes, definition information about an initial medical validation model; performing, by the master node, a federated learning process together with the plurality of computing nodes, to jointly train the initial medical validation model using respective processed local training datasets available at the plurality of computing nodes, the respective local training datasets being processed by the plurality of computing nodes based on the definition information; and determining, by the master node, a final medical validation model based on a result of the federated learning process.

In some embodiments, the method further comprises: distributing, by the master node, the final medical validation model to at least one of the plurality of computing nodes or at least one further computing node for use in medical validation.

In some embodiments, the respective local training datasets comprise historical medical data generated in medical tests and labeling information indicating local validation categories of the historical medical data.

In some embodiments, the definition information indicates unified item names in medical data input to the initial medical validation model, and unified validation categories output from the initial medical validation model, the unified validation categories indicating a plurality of predetermined validation actions to be performed on the medical data. In some embodiments, the respective local training datasets are processed by mapping local item names used in the historical medical data to the unified item names, and mapping the local validation categories to the unified validation categories.

In some embodiments, the definition information further indicates a scaled value range for an item in medical data input to the initial medical validation model. In some embodiments, the respective local training datasets are processed by mapping values of the item in the historical medical data into values within the scaled value range.

In some embodiments, the definition information further indicates a unified red flag rule for medical data prevented from being input to the initial medical validation model. In some embodiments, the respective local training datasets are processed by filtering out historical medical data satisfying the unified red flag rule.

In some embodiments, the definition information indicates an item in medical data input to the initial medical validation model, and a value of the indicated item is unavailable from historical medical data in a local training dataset. In some embodiments, the local training dataset are processed by filling in a predetermined value for the indicated item.

In some embodiments, the predetermined value comprises either one of an average value of a reference value range of the indicated item and a median value of available values of the indicated item in historical medical data generated in other medical tests.

In some embodiments, determining the final medical validation model to at least one of the plurality of computing nodes comprises: obtaining, by the master node, a trained medical validation model from the result of the federated learning process; distributing the trained medical validation model to the plurality of computing nodes; receiving feedback from the plurality of computing nodes, the feedback indicating respective performance metrics of the trained medical validation model determined by the computing nodes using respective local validation datasets; and determining the final medical validation model based on the received feedback.

In some embodiments, determining the final medical validation model based on the received feedback comprises: in response to the respective performance metrics meeting a model release criterion, determining the trained medical validation model as the final medical validation model; and in response to the respective performance metrics failing to meet the model release criterion, adjusting the trained medical validation model to generate the final medical validation model.

In some embodiments, the master node is communicatively connected with the plurality of computing nodes in a star topology network.

In a second aspect, example embodiments of the present disclosure provide a computer-implemented method. The method comprises receiving, by a computing node and from a master node, definition information about an initial medical validation model; processing a local training dataset at least based on the definition information; and performing a federated learning processing together with the master node and at least one further computing node, to jointly train the initial medical validation model using the processed local training dataset.

In some embodiments, the method further comprises: receiving, by the computing node and from the master node, a final medical validation model determined from the federated learning process.

In some embodiments, the local training dataset comprises historical medical data generated in medical tests and labeling information indicating local validation categories of the historical medical data.

In some embodiments, the definition information indicates unified item names in medical data input to the initial medical validation model, and unified validation categories output from the initial medical validation model, the unified validation categories indicating a plurality of predetermined validation actions to be performed on the medical data. In some embodiments, processing the local training dataset comprises: mapping local item names used in the historical medical data to the unified item names, and mapping the local validation categories to the unified validation categories.

In some embodiments, the definition information further indicates a scaled value range for an item in medical data input to the initial medical validation model. In some embodiments, processing the local training dataset comprises: mapping values of the item in the historical medical data into values within the scaled value range.

In some embodiments, the definition information further indicates a unified red flag rule for medical data prevented from being input to the initial medical validation model. In some embodiments, processing the local training dataset comprises: filtering historical medical data satisfying the unified red flag rule out from the local training dataset.

In some embodiments, the definition information indicates an item in medical data input to the initial medical validation model, and a value of the indicated item is unavailable from historical medical data generated in a medical test. In some embodiments, processing the local training dataset comprises: processing the historical medical data by filling in a predetermined value for the indicated item.

In some embodiments, the predetermined value comprises either one of an average value of a reference value range of the indicated item and a median value of available values of the indicated item in historical medical data generated in other medical tests.

In some embodiments, the method further comprises: receiving, from the master node, a trained medical validation model determined from a result of the federated learning process; determining a performance metric of the trained medical validation model using a local validation datasets; and transmitting, to the master node, feedback indicating the determined performance metric.

In a third aspect, example embodiments of the present disclosure provide an electronic device. The electronic device comprises at least one processor; and at least one memory comprising computer readable instructions which, when executed by the at least one processor of the electronic device, cause the electronic device to perform the steps of the method in the first aspect described above.

In a fourth aspect, example embodiments of the present disclosure provide an electronic device. The electronic device comprises at least one processor; and at least one memory comprising computer readable instructions which, when executed by the at least one processor of the electronic device, cause the electronic device to perform the steps of the method in the second aspect described above.

In a fifth aspect, example embodiments of the present disclosure provide a computer program product comprising instructions which, when executed by a processor of an apparatus, cause the apparatus to perform the steps of any one of the methods in the first aspect described above.

In a sixth aspect, example embodiments of the present disclosure provide a computer program product comprising instructions which, when executed by a processor of an apparatus, cause the apparatus to perform the steps of any one of the methods in the second aspect described above.

In a seventh aspect, example embodiments of the present disclosure provide a computer readable medium comprising program instructions for causing an apparatus to perform at least the method in the first aspect described above. The computer readable medium may be a non-transitory computer readable medium in some embodiments.

In an eighth aspect, example embodiments of the present disclosure provide a computer readable medium comprising program instructions for causing an apparatus to perform at least the method in the second aspect described above. The computer readable medium may be a non-transitory computer readable medium in some embodiments.

Generally, various example embodiments of the present disclosure may be implemented in hardware or special purpose circuits, software, logic or any combination thereof. Some aspects may be implemented in hardware, while other aspects may be implemented in firmware or software which may be executed by a controller, microprocessor or other computing device. While various aspects of the example embodiments of the present disclosure are illustrated and described as block diagrams, flowcharts, or using some other pictorial representations, it will be appreciated that the blocks, apparatuses, systems, techniques, or methods described herein may be implemented in, as non-limiting examples, hardware, software, firmware, special purpose circuits or logic, general purpose hardware or controller or other computing devices, or some combination thereof.

The present disclosure also provides at least one computer program product tangibly stored on a non-transitory computer readable storage medium. The computer program product includes computer-executable instructions, such as those included in program modules, being executed in a device on a target real or virtual processor, to carry out the methods/processes as described above. Generally, program modules include routines, programs, libraries, objects, classes, components, data structures, or the like that perform particular tasks or implement particular abstract data types. The functionality of the program modules may be combined or split between program modules as desired in various embodiments. Computer-executable instructions for program modules may be executed within a local or distributed device. In a distributed device, program modules may be located in both local and remote storage media.

The computer readable medium may be a computer readable signal medium or a computer readable storage medium. A computer readable medium may include but is not limited to an electronic, magnetic, optical, electromagnetic, infrared, or semiconductor system, apparatus, or device, or any suitable combination of the foregoing. More specific examples of the computer readable storage medium would include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing.

Computer program code for carrying out methods disclosed herein may be written in any combination of one or more programming languages. The program code may be provided to a processor or controller of a general purpose computer, special purpose computer, or other programmable data processing apparatus, such that the program codes, when executed by the processor or controller, cause the functions/operations specified in the flowcharts and/or block diagrams to be implemented. The program code may execute entirely on a computer, partly on the computer, as a stand-alone software package, partly on the computer and partly on a remote computer or entirely on the remote computer or server. The program code may be distributed on specially-programmed devices which may be generally referred to herein as “modules”. Software component portions of the modules may be written in any computer language and may be a portion of a monolithic code base, or may be developed in more discrete code portions, such as is typical in object-oriented computer languages. In addition, the modules may be distributed across a plurality of computer platforms, servers, terminals, mobile devices and the like. A given module may even be implemented such that the described functions are performed by separate processors and/or computing hardware platforms.

While operations are depicted in a particular order, this should not be understood as requiring that such operations be performed in the particular order shown or in sequential order, or that all illustrated operations be performed, to achieve desirable results. In certain circumstances, multitasking and parallel processing may be advantageous. Likewise, while several specific implementation details are contained in the above discussions, these should not be construed as limitations on the scope of the present disclosure, but rather as descriptions of features that may be specific to particular embodiments. Certain features that are described in the context of separate embodiments may also be implemented in combination in a single embodiment. Conversely, various features that are described in the context of a single embodiment may also be implemented in multiple embodiments separately or in any suitable sub-combination.

Although the present disclosure has been described in languages specific to structural features and/or methodological acts, it is to be understood that the present disclosure defined in the appended claims is not necessarily limited to the specific features or acts described above. Rather, the specific features and acts described above are disclosed as example forms of implementing the claims.

1. A computer-implemented method, comprising:
transmitting, by a master node to a plurality of computing nodes, definition information about an initial medical validation model;
performing, by the master node, a federated learning process together with the plurality of computing nodes, to jointly train the initial medical validation model using respective processed local training datasets available at the plurality of computing nodes, the respective local training datasets being processed by the plurality of computing nodes based on the definition information; and
determining, by the master node, a final medical validation model based on a result of the federated learning process.

2. The method of claim 1, further comprising:
distributing, by the master node, the final medical validation model to at least one of the plurality of computing nodes or at least one further computing node for use in medical validation.

3. The method of claim 1, wherein the respective local training datasets comprise historical medical data generated in medical tests and labeling information indicating local validation categories of the historical medical data.

4. The method of claim 3, wherein the definition information indicates unified item names in medical data input to the initial medical validation model, and unified validation categories output from the initial medical validation model, the unified validation categories indicating a plurality of predetermined validation actions to be performed on the medical data; and
wherein the respective local training datasets are processed by mapping local item names used in the historical medical data to the unified item names, and mapping the local validation categories to the unified validation categories.

5. The method of claim 3, wherein the definition information further indicates a scaled value range for an item in medical data input to the initial medical validation model, and
wherein the respective local training datasets are processed by mapping values of the item in the historical medical data into values within the scaled value range.

6. The method of claim 2, wherein the definition information further indicates a unified red flag rule for medical data prevented from being input to the initial medical validation model, and
wherein the respective local training datasets are processed by filtering out historical medical data satisfying the unified red flag rule.

7. The method of claim 2, wherein the definition information indicates an item in medical data input to the initial medical validation model, a value of the indicated item being unavailable from historical medical data in a local training dataset, and
wherein the local training dataset are processed by filling in a predetermined value for the indicated item.

8. The method of claim 7, wherein the predetermined value comprises either one of an average value of a reference value range of the indicated item and a median value of available values of the indicated item in historical medical data generated in other medical tests.

9. The method of claim 1, wherein determining the final medical validation model comprises:
obtaining, by the master node, a trained medical validation model from the result of the federated learning process;
distributing the trained medical validation model to the plurality of computing nodes;
receiving feedback from the plurality of computing nodes, the feedback indicating respective performance metrics of the trained medical validation model determined by the computing nodes using respective local validation datasets; and
determining the final medical validation model based on the received feedback.

10. The method of claim 9, wherein determining the final medical validation model based on the received feedback comprises:
in response to the respective performance metrics meeting a model release criterion, determining the trained medical validation model as the final medical validation model; and
in response to the respective performance metrics failing to meet the model release criterion, adjusting the trained medical validation model to generate the final medical validation model.

11. The method of claim 1, wherein the master node is communicatively connected with the plurality of computing nodes in a star topology network.

12-21. (canceled)

22. An electronic device comprising:
at least one processor; and
at least one memory comprising computer readable instructions which, when executed by the at least one processor of the electronic device, cause the electronic device to perform the steps of claim 1.

23. (canceled)

24. A computer program product comprising instructions which, when executed by a processor of an apparatus, cause the apparatus to perform the steps of claim 1.

25. (canceled)
